(19) United States
(12) Patent Application Publication
(10) Pub. No.: US 2007/0141148 A1
(43) Pub. Date: Jun. 21, 2007

(54) Neramexane MR matrix tablet

(75) Inventors: Bernhard Hauptmeier, Gelnhausen (DE); Andreas Becker, Bensheim (DE)

Correspondence Address: THE FIRM OF HUESCHEN AND SAGE, SEVENTH FLOOR, KALAMAZOO BUILDING, 107 WEST MICHIGAN AVENUE, KALAMAZOO, MI, 49007, US

(73) Assignee: MERZ PHARMA GMBH & CO. KGAA, FRANKFURT, MAIN (DE)

(21) Appl. No.: 11/604,986

(22) Filed: Nov. 27, 2006

Related U.S. Application Data
(60) Provisional application No. 60/740,975, filed on Nov. 30, 2005.

(51) Int. Cl. A61K 9/22; A61K 31/13

(52) U.S. Cl. 424/468; 514/579

(57) ABSTRACT

The invention provides novel oral modified release dosage forms of neramexane which are useful for the continuous therapy of patients suffering from diseases and conditions such as Alzheimer's dementia and neuropathic pain. The compositions have drug release profiles that are suitable for achieving steady state plasma concentrations of neramexane which have relatively small fluctuation when administered on a twice-daily or even once-daily regimen. The dosage forms may be designed as modified release matrix tablets, which are optionally coated for taste masking. The invention further provides therapeutic methods of treating conditions such as Alzheimer's dementia and neuropathic pain which involve the administration of such dosage forms.

### FIELD OF THE INVENTION

The invention relates to pharmaceutical dosage forms, in particular to modified release dosage forms suitable for oral administration. In another aspect, the invention relates to novel uses of the active compound neramexane and therapeutic methods involving such uses.

### BACKGROUND OF THE INVENTION

Neramexane, also known as 1-amino-1,3,3,5,5-pentamethylcyclohexane, is a member of the class of orally active 1-aminocyclohexanes, and has been found to be useful in the therapy of various diseases especially in certain neurological diseases, including Alzheimer's disease and neuropathic pain. The compound and its derivatives are disclosed in detail in U.S. Pat. Nos. 6,034,134 and 6,071,966, the subject matter of which patents is hereby incorporated by reference. It is believed that the therapeutic action of neramexane is related to the inhibition of the effects of excessive glutamate at the N-methyl-D-aspartate (NMDA) receptors of nerve cells, for which reason the compound is also categorized as an NMDA antagonist, or NMDA receptor antagonist. More specifically, neramexane appears to be a low to moderate-affinity, non-competitive NMDA-receptor antagonist believed to selectively block the excitotoxic effects associated with abnormal transmission of glutamate, which is a neurotransmitter that performs an integral role in the neural pathways associated with learning and memory, and which is believed to play a role in Alzheimer's disease.

Neramexane appears to be therapeutically effective after oral administration. In clinical trials, it may be orally administered in the form of immediate release dosage forms. Typically, neramexane is administered at least twice a day during continuous therapy in order to ensure that therapeutically effective plasma concentrations are maintained.

Modified release solid oral dosage forms permit the modified release of the active ingredient over an extended period of time in an effort to maintain therapeutically effective plasma levels over similarly extended time intervals and/or to modify other pharmacokinetic properties of the active ingredient. Immediate release solid dosage forms permit the release of most or all of the active ingredient over a short period of time, such as 60 minutes or less, and make rapid absorption of the drug possible. A multiphase release profile (i.e., a composition containing at least an immediate release formulation and at least one modified release formulation) may be employed to attain one or more combinations of release rates to attain more specific therapeutic objectives such as a portion of drug releasing immediately, followed by an extended release. However, modulation of the release rate of an active ingredient does not necessarily ensure that long-lasting effective blood level concentrations will be consistently achieved or that the pharmacological effect will be based solely on the release of the drug.

It has been found that a low frequency of administration, such as once-daily dosing, is desirable for most continuous drug therapies. In many studies investigating patient compliance and dosing frequency, a negative correlation between the two parameters was found. It is also believed that particularly those patients who suffer from dementia may find it difficult to adhere to a therapeutic regimen requiring several dosings every day.

A general method of modified release for N-methyl-D-aspartate (NMDA) receptor antagonists was described in U.S. Pat. No. 6,194,000. This method also involves preparing an instant release component and a modified release component to arrive at the final formulation. The patent discloses a pellet (not a bead) consisting of a coated core, the coating being any suitable coating using organic solvent-based systems. However, not all NMDA antagonists act in the same manner, and this patent does not specifically disclose compositions containing neramexane.

Currently, a dosing regimen of neramexane of twice a day is employed using immediate release tablets. This may be undesirable because patient compliance decreases as the frequency of taking a drug increases. Moreover, administration of an immediate-release tablet can lead to greater frequency of adverse events due to a faster rate of absorption. For pain treatment, it is very important to maintain the pain relief without additional discomfort. There is therefore an existing and continual need for a once a day modified release formulation containing neramexane or a pharmaceutically acceptable salt of neramexane with reliable slower absorption over a targeted period of time.

While there is a need for modified release dosage forms suitable for the administration of neramexane, which appears to be useful in the treatment of certain Alzheimer's dementia patients, such dosage forms have not been described or successfully developed. The development of a modified release dosage form for neramexane is challenging due to the high solubility of the molecule in aqueous media over a wide pH—range. In particular, there is a need for modified release dosage forms of neramexane which are suitable for once-daily administration, and which are well-tolerated. Furthermore, there is a need for modified release dosage forms of neramexane which are robust, and whose dissolution behavior does not depend on the state of digestion or dosage form transit through the gastrointestinal tract.

These and other needs are addressed by the present invention which is disclosed in the subsequent description, examples, and claims.

### SUMMARY OF THE INVENTION

In a first aspect, the invention provides dosage forms for the oral administration of neramexane, a novel NMDA antagonist which has been found useful for the treatment of Alzheimer's disease (including mild, moderate, or severe Alzheimer's dementia) and other disorders. The dosage forms have modified release characteristics and are suitable for continuous therapeutic dosing regimen. High plasma concentration peaks are avoided.

The slow release of the active ingredient over an extended period of time leads to lower peak concentrations at the beginning of the dosing as well as at steady state and to a slower absorption. The slower absorption is achieved when the dissolution rate is slower than the absorption and thus the dissolution becomes the pace making step. The deceleration of absorption can be expected to improve the tolerability of the active ingredient.

In a further embodiment, the dosage forms of the invention are designed as tablets comprising the active ingredient dispersed within a matrix formed by at least one release-controlling excipient, and optionally one or more further pharmaceutically acceptable excipients. The dosage forms exhibit a dissolution time of at least about 1 hour for a dose fraction of 10 to 70 wt.-% of the active compound incorporated therein.

Furthermore, the invention provides an oral modified release dosage form comprising a therapeutically effective amount of an active compound which is highly soluble in aqueous media, selected from neramexane and pharmaceutically acceptable salts, solvates, isomers, conjugates, prodrugs and derivatives thereof, and at least one release-controlling excipient, wherein the content of the excipient is selected to achieve an in vitro active compound release profile characterized by a dissolution time of at least about 1 hour for a fraction of 50 wt.-% of the amount of the active compound.

According to another aspect, the invention provides oral modified release dosage forms of neramexane which comprise at least one release-controlling excipient, and wherein the release-controlling excipient is selected to achieve an in vitro drug dissolution profile which is substantially independent of the pH of the dissolution medium.

In a further aspect, oral modified release dosage forms of neramexane in the form of compressed tablets are provided. The tablets comprise at least one release-controlling excipient which is selected to achieve a drug dissolution profile that is substantially independent of the hardness of the tablet.

In a further aspect, oral modified release dosage forms of neramexane in the form of compressed tablets are provided. The tablets comprise at least one release-controlling excipient which is selected to achieve a drug dissolution profile that is substantially independent over a wide range of agitation of the dissolution medium.

In yet a further aspect, oral modified release dosage forms of neramexane are provided which exhibit a low fluctuation index of neramexane plasma concentrations in the steady state upon once-daily administration. In particular, the fluctuation index is about 0.4 or less.

Furthermore, uses of neramexane and methods of treatment involving the twice-daily or once-daily administration of the dosage forms of the invention are provided. The methods may be useful for the therapy of mild, moderate, or severe Alzheimer's dementia, or neuropathic pain. Further, the methods may be useful for the therapy of diabetic neuropathic pain, amyotrophic lateral sclerosis, multiple sclerosis, irritable bowel syndrome, appetite disorders, obesity, binge eating disorders, autism, attention deficit syndrome, attention deficit hyperactivity disorder, bipolar disorder, tinnitus, mycosis, or psoriasis.

Moreover, the methods may be useful for the therapy of conditions associated with cognitive impairment such as dementia, neurodegenerative dementia, mild, moderate and severe Alzheimer's dementia, Parkinson's dementia, AIDS dementia, schizophrenia, attention deficit syndrome, attention deficit hyperactivity disorder, Korsakoff syndrome, cerebrovascular dementia, frontotemporal dementia, autism, corticobasal degeneration including corticobasal degeneration dementia, Lewis body disease, mild cognitive impairment, dementia due to inflammation or infection, multiple sclerosis, or amyotrophic lateral sclerosis.

Further aspects of the invention will become obvious on the basis of the following detailed description and the patent claims.

### DETAILED DESCRIPTION OF THE INVENTION

The invention provides an oral modified release dosage form comprising a therapeutically effective amount of a highly soluble active compound in aqueous media and at least one release-controlling excipient. The active compound is selected from neramexane and pharmaceutically acceptable salts, solvates, isomers, conjugates, prodrugs and derivatives thereof. The content of the rate-controlling excipient is selected to achieve an in vitro drug release profile which is characterized by a dissolution time of at least about 1 hour for a fraction of 10 to 70 wt.-%, such as 50 wt.-% of the amount of the active compound present in the dosage form.

Neramexane, may be used according to the invention in the form of any of its pharmaceutically acceptable salts, solvates, isomers, conjugates, prodrugs and derivatives, any references to neramexane in this description should be understood as also referring to such salts, solvates, isomers, conjugates, prodrugs and derivatives.

In one embodiment of the invention, neramexane is incorporated in the dosage form of the invention in form of one of its salts, such salts generally having substantial water solubility.

Potentially suitable salts of neramexane include, but are not limited to, acid addition salts, such as those made with hydrochloric, methylsulfonic, hydrobromic, hydroiodic, perchloric, sulfuric, nitric, phosphoric, acetic, propionic, glycolic, lactic pyruvic, malonic, succinic, fumaric, tartaric, citric, benzoic, carbonic cinnamic, mandelic, methanesulfonic, ethanesulfonic, hydroxyethanesulfonic, benezenesulfonic, p-toluene sulfonic, cyclohexanesulfamic, salicyclic, p-aminosalicylic, 2-phenoxybenzoic, and 2-acetoxybenzoic acid.

A therapeutically effective dose of neramexane is defined in consideration of factors such as the specific condition which is to be treated, the weight of the patient, the patient's condition, the dosing regimen etc. It is currently believed that a cumulated daily oral dose of approx. 5 to about 150 mg, such as from about 5 mg to about 120 mg or from approx. 5 mg to 100 mg of neramexane or of a salt of neramexane such as neramexane mesylate, is therapeutically effective for the treatment of at least some of the conditions for which neramexane appears to be useful. A cumulated daily oral dose of about 10 mg to about 90 mg of neramexane or of a salt of neramexane such as neramexane mesylate may be further preferred.

Moreover, a cumulated daily dose of about 5 mg to about 50 mg, such as 5 mg, 6.25 mg, 7.5 mg, 10 mg, 12.5 mg, 15 mg, 17.5 mg, 20 mg, 22.5 mg, 25 mg, 27.5 mg, 30 mg, 32.5 mg, 35 mg, 37.5 mg, 40 mg, 42.5 mg, 45 mg, 47.5 mg and 50 mg of neramexane mesylate or an equimolar amount of neramexane, another pharmaceutically acceptable salt, a solvate, an isomer, a conjugate, a prodrug or a derivative thereof, such as neramexane hydrochloride, is therapeutically effective and simultaneously avoids excessive side effects. Furthermore, a cumulated daily dose of about 5 mg to about 40 mg or of about 10 mg to about 30 mg of neramexane mesylate or an equimolar amount of neramexane, another pharmaceutically acceptable salt, a solvate, an isomer, a conjugate, a prodrug or a derivative thereof may also be useful. The cited amounts and ranges of active compound are useful in treating or alleviating conditions including cognitive impairment and further conditions associated with cognitive impairment (e.g., dementia; neurodegenerative dementia; mild, moderate, or severe Alzheimer's dementia; Parkinson's dementia; AIDS dementia; schizophrenia; attention deficit syndrome; attention deficit hyperactivity disorder; Korsakoff syndrome; cerebrovascular dementia; frontotemporal dementia; autism; corticobasal degeneration including corticobasal degeneration dementia; Lewis body disease; mild cognitive impairment; dementia due to inflammation or infection; multiple sclerosis; or amyotrophic lateral sclerosis). If the modified release solid oral dosage forms are intended for twice daily administration the given amounts of active ingredient may be halved. A lower or higher dose may also be appropriate and therapeutically effective in treating other conditions.

As used herein, a modified release dosage form is a dosage form from which an incorporated active compound is released slowly over a period of time which is substantially longer than about 15 minutes and shorter than 24 hours, and typically over a period of at least about 4 to 12 hours, as determined according to well-established and commonly accepted methods, e.g. by in-vitro dissolution testing according to the U.S. Pharmacopeia, USP 28, or the European Pharmacopeia, EP 5, using typical buffers with pH ranges of 1.0 to 7.2 as dissolution media. This definition is independent of the shape of the release profile, i.e. whether linear, curved according to first-order, second-order, or square root of time-kinetics, sigmoidal, etc. Accordingly, modified release should be understood to include extended release, prolonged release, sustained release, slow release and similar expressions for related drug release characteristics.

In one embodiment, the dosage form of the invention is a formulation that releases neramexane in a non-linear manner over a period of at least about 6 hours, with a release rate which decreases over time. In another embodiment, neramexane is released in a substantially linear manner over at least 6 hours. The dissolution time for 50 wt.-% of the incorporated dose of the active compound to be released is typically at least 1 hour, and may be at least 1.5 hours.

In another embodiment the dissolution time for 40 wt.-% of the incorporated dose of the active compound to be released is typically at least 1 hour, and may be at least 1.5 hours.

In another embodiment the dissolution time for 60 wt.-% of the incorporated dose of the active compound to be released is typically at least 1 hour, and may be at least 1.5 hours.

In another embodiment the dissolution time for 10 to 70 wt.-% of the incorporated dose of the active compound to be released is between about 1 and 8 hours.

In a further embodiment, the release is non-linear, and the dissolution time for 50 wt.-% of the incorporated dose of the active compound to be released is between about 1 and 5 hours, or between about 1 and 4 hours, or between about 1.5 and 3 hours. In contrast, if the release profile is substantially linear, then the dissolution time for 50 wt.-% of the dose is at least about 2 hours, or at least about 3 hours, such as about 4 hours to about 8 hours.

In one embodiment of the invention the in vitro active compound release profile is characterized by a dissolution time ranging from about 1 hour to about 3 hours for a fraction of 50 wt.-% of the amount of the active compound.

A non-linear release profile which is suitable for twice-daily and particularly once-daily dosing is further characterized by a dissolution time of 4 hours for a dose fraction ranging from about 50 wt.-% to about 95 wt.-% amount of the active compound.

In another embodiment, the dose fraction released after 4 hours ranges from about 65 wt.-% to about 95 wt.-%. In another embodiment, the dose fraction released after 4 hours ranges from about 55 wt.-% to about 85 wt.-%. Alternatively, the dose fraction released after 4 hours ranges from about 70 wt.-% to about 85 wt.-%. It has been found that such release behavior is useful for achieving and maintaining therapeutic plasma concentrations of neramexane in the steady state even with a single daily administration.

In another embodiment, a dose fraction ranging from about 75 wt.-% to about 95 wt.-% is released after a dissolution time of 6 hours, such as from about 80 wt.-% to about 90 wt.-%.

It has been found that modified release dosage forms which exhibit drug release profiles as described above are particularly suitable for continuous therapy with neramexane, such as continuous therapy of patients suffering from conditions and disorders selected from mild, moderate or severe Alzheimer's dementia, and neuropathic pain. Furthermore, conditions such as diabetic neuropathic pain, amyotrophic lateral sclerosis, multiple sclerosis, irritable bowel syndrome, appetite disorders, obesity, binge eating disorders, autism, bipolar disorder, attention deficit syndrome, attention deficit hyperactivity disorder, tinnitus, mycosis and psoriasis might be treatable by the modified release dosage forms which exhibit drug release profiles as described above.

The dosage forms are particularly useful for a therapeutic dosing regimen involving continuous twice-daily or once-daily administration.

As used herein, continuous therapy is understood as a period of regular treatment over a time span of at least about 2 weeks, and often for at least about one month. The dosage form of the invention is also suitable for a continuous therapy over several months or even years as it provides the active principle, neramexane, to the patient in a well-tolerated manner, producing therapeutically effective steady state plasma levels with only moderate fluctuation.

Twice-daily and once-daily dosing regimens, as understood herein, involve the repeated administration of an active compound at approximately regular time intervals. Typically, the time of administration does not differ by more than a few hours from day to day. Particularly in the case of a once-daily dosing regimen, relatively even plasma profiles with moderate fluctuations are achieved only if the daily time of administration is similar, e.g. always in the morning, or always in the evening, and does not vary from one day to the next by a difference of more than 3 or 4 hours.

As used herein, steady state means that a regular dosing regimen has been followed for a sufficiently long period of time that the average plasma concentration of the active compound after administration is similar to the average plasma concentration after the previous administration. Similarly, the peak and trough plasma concentrations are similar to the respective concentrations after the previous dosing.

The time to reach steady state depends primarily on the elimination half life of the active principle. After 4 elimination half lives, repeated dosing at the same time intervals typically leads to average plasma concentrations which are about 93-94 % of the average steady state plasma concentrations. Given the considerable biological variability even within the same individuals, it may be assumed that after 4-5 half lives, plasma concentrations are practically the same as steady state plasma concentrations.

The use of the dosage form of the invention may comprise continuous therapy with once-daily administration. Once-daily administration of an immediate release formulation of neramexane leads to plasma concentration fluctuations which could increase the risk of side effects through high peak levels and/or increase the risk of achieving only sub-therapeutic trough concentrations. The fluctuation index of plasma concentrations, according to this once-daily regimen using conventional formulations, is in the region of 0.4 to 0.5, which is about twice as high as in a twice-daily dosing regimen with immediate release formulations.

As used herein, the fluctuation index expresses the fluctuation between the peak and the trough concentration relative to the average plasma concentration:

$${I}_{F} = \frac{{c}_{ss ( max )} - {c}_{ss ( min )}}{{c}_{ss ( av )}}$$

wherein I_{F} is the fluctuation index, c_{SS(max)} is the peak plasma concentration at steady state, C_{SS(min)} is the trough plasma concentration at steady state, and C_{SS(av)} is the mean or average plasma concentration at steady state. It is currently believed that a fluctuation index of more than about 0.45 is not particularly useful in the continuous therapy with neramexane. According to the invention, the release profile of neramexane from the dosage form is tailored to yield a fluctuation index of not more than about 0.45 during continuous once-daily therapy in steady state, which is achieved by making appropriate selections with regard to the nature, grade, and relative quantity of the release-controlling excipient (or excipients) according to the guidance provided herein. Typically, the fluctuation index is not more than about 0.4 during continuous once-daily therapy. In further embodiments, the fluctuation index achieved with the dosage form of the invention is not more than about 0.38 or even not more than about 0.35, again applying the same therapeutic regimen.

The release behavior of the dosage form of the invention can be achieved by various means, such as by several types of dosage form designs and formulation strategies. It is not considered crucial by which mechanism the drug release is controlled. For example, drug release may be controlled by the diffusion of neramexane through a diffusion barrier, such as through a polymeric film, by diffusion through a matrix, by erosion of a matrix in which the active compound is embedded or dispersed, or by a combination of more than one of these mechanisms.

For example, the dosage form may be designed and formulated as a coated solid single-unit dosage form, such as a coated tablet, wherein the coating functions as a diffusion barrier and provides for prolonged drug release. In this case, the coating contains at least one release-controlling excipient whose quality and quantity is selected to achieve the above-described release properties.

As used herein, an excipient is a pharmaceutically acceptable, physiologically inactive ingredient of a dosage form. A release-controlling excipient is an excipient which is capable of substantially decreasing the release rate of an active compound from a dosage form or formulation. If the coating of a tablet is designed to achieve the modified release characteristics, the release-controlling excipient in the coating is typically a water-insoluble polymer. Potentially suitable polymers are generally known to the person trained in the field of pharmaceutical formulations. Examples of such polymers include alginates, ethylcellulose, cellulose acetate, cellulose acetate butyrate, methacrylate ester copolymer, polyoxyethylenoxide polymers, zein, polyvinyl acetate-polyvinylpyrrolidone copolymer and the like. The polymers may be provided as organic solutions or aqueous dispersions and sprayed onto tablets using conventional coating equipment. Typically, the coating solution or dispersion will also contain a plasticizer, such as glycerol, propylene glycol, polyethylene glycol, diethyl phthalate, dibutyl phthalate, dibutyl sebacate, triacetin, triethyl citrate, acetyltriethyl citrate, acetyltributyl citrate, castor oil, mono- and diglycerides etc. Examples of further optional excipients are pigments, flavors, sweeteners, opacifiers, and antiadhesives.

According to a typical embodiment, however, the dosage form of the invention is designed as a solid modified release matrix in which the active ingredient is embedded or dispersed, and from which it is released slowly over an extended period of time. In this case, no modified release coating is necessary, and at least one release-controlling excipient is comprised in the matrix.

Typically, the matrix does not disintegrate rapidly in aqueous media at physiological temperatures. Active compound release from the matrix may be controlled by the diffusion of the active compound through the matrix, by the erosion of the matrix, or both.

In one embodiment, the matrix is designed to be a compressed tablet. In order that the matrix-type tablet does not disintegrate rapidly but effects modified active compound release, it comprises at least one release-controlling excipient, which is preferably selected from the group consisting of water-insoluble lipids and waxes, water-insoluble polymers, and water-swellable polymers: If more than one release-controlling excipient is present in the matrix, it is also possible to combine excipients from different chemical subgroups.

The release-controlling excipient or mixture of excipients is selected in an amount sufficient to achieve the release characteristics described herein-above. Depending on its type, the typical content of the excipient in the solid matrix is from about 10 wt.-% to about 80 wt.-%.

Suitable release-controlling water-insoluble polymers include, for example, ethyl cellulose, cellulose acetate, cellulose acetate butyrate, methacrylate ester copolymer, zein, polyvinyl acetate, and polyvinyl acetate-polyvinylpyrrolidone copolymer. Suitable release-controlling lipids and waxes include, for example, beeswax, natural or synthetic mono-, di- and triglycerides of medium and long chain fatty acids such as hydrogenated vegetable oils, carnauba wax, petroleum wax, microcrystalline wax, long chain fatty acids, long chain fatty alcohols, esters of fatty acids and fatty alcohols etc. Further examples of pharmaceutically acceptable water-insoluble polymers, lipids and waxes which may also be employed in the formulation of a matrix are well-known to the person skilled in pharmaceutics.

Depending upon the hydrophilic (erodable or non-erodable) or hydrophobic nature of the matrix, the matrix may be a material that swells upon contact with gastric fluid to a size that is large enough to promote retention in the stomach while the subject is in the digestive state. In addition to these diffusion based matrices, the matrix may also be in an erodable form. The digestive state is induced by food ingestion and begins with a rapid and profound change in the motor pattern of the upper gastrointestinal (GI) tract. The change consists of a reduction in the amplitude of the contractions that the stomach undergoes and a reduction in the pyloric opening to a partially closed state. The result is a sieving process that allows liquids and small particles to pass through the partially open pylorus while indigestible particles that are larger than the pylorus are retropelled and retained in the stomach. In other words, biological fluids migrate through the matrix and dissolve the active ingredient which is released by diffusion through the matrix which, simultaneously, modulates the release rate. The controlled-release matrix in these embodiments of the invention is therefore selected as one that can swell to a size large enough to be retropelled and thereby retained in the stomach, causing the prolonged release of the drug to occur in the stomach rather than in the intestine. Disclosures of oral dosage forms that swell to sizes that will prolong the residence time in the stomach are found in U.S. Pat. Nos. 5,007,790, 5,582,837, and 5,972,389, as well as International (PCT) Patent Application WO 98/55107 and WO 96/26718. Each of the documents cited in this paragraph is incorporated herein by reference in its entirety.

In such an embodiment, the solid matrix is designed as a water-swellable, hydrophilic matrix comprising a release-controlling agent selected from the group of water-swellable polymers. Suitable matrix-forming polymers may be water-soluble or -insoluble. Suitable polymers absorb substantial amounts of water when placed into contact with aqueous media which typically results in the formation of an aqueous gel. The strength of the aqueous gel depends on the type and quantity of polymer and on the presence of other compounds in the matrix. Drug release may occur through diffusion of the active compound through aqueous micropores or microchannels within the three-dimensional polymeric gel network, and also through continuous erosion or disintegration of the most superficial gel layers of the matrix.

Usually, the gel formation in a hydrophilic modified release matrix is a process which starts in the outer regions of the tablet and progresses slowly towards the core. Thus, it is believed that several zones or layers may be coexisting in such matrix during drug release, i.e. a non-hydrated core region, an intermediate gel layer surrounding the core region, and an eroding outer region. However, these considerations should be appreciated as a theoretical model only; they should not be construed to limit the scope of any subject matter claimed herein.

Among the suitable water-swellable polymers according to the invention are cellulose polymers and their derivatives including, but not limited to, methylcellulose, hydroxymethylcellulose, hydroxyethylcellulose, hydroxypropylcellulose, hydroxybutylcellulose, hydroxyethyl methylcellulose, hydroxypropyl methylcellulose, carboxymethylcellulose, carboxymethyl ethylcellulose, and microcrystalline cellulose polysaccharides and their derivatives, polyalkylene oxides, polyethylene glycols, chitosan, alginate, carrageen, galactomannan, tragacanth, agar, acacia, guar gum, xanthan gum, pectin, carboxymethyl amylopectin, chitosan, maleic anhydride copolymers, polyacrylate, polymethacrylate, methacrylate copolymer, polyvinyl alcohol, polyvinylpyrrolidone, polyvinylpyrrolidone vinyl acetate copolymer, poly(2-ethyl-2-oxazoline), poly(ethyleneimine), polyurethane hydrogels, crosslinked polyacrylic acids and their derivatives, and mixtures of any of these.

Further examples are copolymers of the polymers listed above, including block copolymers and graft polymers. Specific examples of copolymers are PLURONIC® and TECTONIC®, which are polyethylene oxide-polypropylene oxide block copolymers available from BASF Corporation, Chemicals Div., Wyandotte, Mich., USA. Further examples are hydrolyzed starch polyacrylonitrile graft copolymers, commonly known as “Super Slurper” and available from Illinois Corn Growers Association, Bloomington, Ill., USA.

Among the above-mentioned water-swellable polymers, some may be considered non-ionic polymers, such as non-ionic cellulose ether. An example of such polymer is hydroxypropyl methylcellulose (HPMC) also called hypromellose, which may be used alone or in combination with other polymers.

It has been surprisingly found that hydroxypropyl methylcellulose is, even without the addition of another release-controlling excipient, capable of forming a swellable matrix from which a highly water-soluble form of neramexane, such as neramexane mesylate, is released over a prolonged period of time. This is in contrast to the common assumption that it is difficult to formulate modified release matrices with substantially water-soluble active agents based on one water-swellable polymer such as hydroxypropyl methylcellulose alone.

Different grades of hydroxypropyl methylcellulose according to the invention include HPMC 2208, HPMC 2906 and HPMC 2910. These grades differ in their degree of substitution both with regard to the methyl(or methoxyl) and hydroxypropyl(or hydroxypropoxyl) groups. In HPMC 2208, an average of approx. 22% (range: 19 to 24%) of the original hydroxyl groups of the cellulose have been reacted into methoxyl groups, and an average of approx. 8% (range: 7 to 12%) have been reacted into hydroxypropoxyl groups. HPMC 2906 comprises approx. 29% methoxyl groups and approx. 6% hydroxypropoxyl groups, and HPMC 2910 comprises approx. 29% methoxyl groups and approx. 10% hydroxypropoxyl groups. A typical grade of hydroxypropyl methylcellulose is HPMC 2208, such as is commercially available as Methocel K 100M CR. This grade of Methocel, is also characterized by a relatively high molecular weight, as indicated by an apparent viscosity of approx. 100,000 cP for an aqueous solution of 2 wt.-% at 20° C.

The relative amount of water-swellable polymer needed to achieve the desired release characteristics depends, inter alia, on the selected polymer type and grade, the presence or absence of other excipients having impact on drug release, and on the desired drug load of the matrix. In the case of hydroxypropyl methylcellulose, the ratio of this polymer to the active compound is typically selected in the range from about 10:1 to about 1:10, and may be from about 5:1 to about 1:5. If a high-viscosity HPMC such as Methocel K 100M CR is chosen as principal release-controlling excipient, a typical ratio between the HPMC and the active compound is from about 4:1 to about 1:4 or from about 2:1 to about 1:2. For example, if the matrix is designed to accommodate a dose of about 50 mg of neramexane mesylate, the content of Methocel K 100M CR is, according to this embodiment, in the region from about 5 mg to about 100 mg, or from about 12.5 mg to about 200 mg.

It has also been found to be useful to combine the drug substance and the water-swellable polymer with a further excipient selected from a class of dry binding agents or compression aids, which are also sometimes referred to as tabletting aids, fillers, diluents, or bulking agents. Such excipients are capable of increasing the internal binding forces of the matrix after compression. Usually they possess a high degree of plastic deformability. Their influence on drug dissolution or drug release may be relatively moderate. Examples of suitable members of this excipient category include anhydrous lactose, lactose monohydrate, calcium phosphate, dibasic calcium phosphate, calcium hydrogen phosphate, calcium sulfate, sucrose, dextrose, mannitol, sorbitol, cellulose, microcrystalline cellulose, and co-processed mixtures of lactose and microcrystalline cellulose (commercially available e.g. as Cellactose). A typical dry binding agent is microcrystalline cellulose, such as the commercially available Avicel PH.

Various types of microcrystalline cellulose are suitable for carrying out the invention. The product grades that are commercially available differ predominantly in particle size and moisture content, and should be selected depending on the method for the preparation of the matrix. For example, it has been found that Avicel PH 102 and certain other Avicel grades are particularly suitable for the preparation of a matrix tablet by direct compression.

The content of the dry binding agent or compression aid in the dosage form of the invention is selected according to various formulation criteria, such as the type and grade of the dry binding agent or compression aid, the type, grade, and quantity of the water-swellable polymer, the active compound load, the presence of further excipients having an impact on compressibility etc. Typically, the content is at least about 10 wt.-% relative to the weight of the matrix, and often at least about 15 wt.-%. In further embodiments, the content is between about 15 wt.-% and about 60 wt.-%, such as between about 15 wt.-% and about 50 wt.-%.

The ratio of the water-swellable polymer and the dry binding agent or compression aid in the matrix is typically in the range from about 6:1 to 1:6, such as from about 5:1 to 1:5, and in particular embodiments from about 3:1 to about 1:3, and from about 2:1 to 1:2, respectively. In another embodiment, the water-swellable polymer is hydroxypropyl methylcellulose, in particular Methocel K 100M CR, and the dry binding agent or compression aid is microcrystalline cellulose, and they are present in the matrix in a ratio from about 2:1 to about 1:2, and at a total content of both excipients in the matrix from about 50 wt.-% to about 85 wt.-%, such as between about 60 wt.-% and about 75 wt.-%.

As indicated above, it is an embodiment of the dosage form that it is designed as a compressed matrix, i.e. a compressed matrix tablet. Various methods are available and suitable for the preparation of such tablets, typical methods being the compression of granules prepared by wet or dry granulation, and the direct compression of powder mixtures into compacts. Both methods are well-known to the skilled person in the technical field.

Wet-granulation methods involve the weighing of ingredients, including the active compound and most of the excipients, plus a liquid binder solution, mixing the ingredients, agglomerating them, screening them damp, drying them, dry screening, lubrication, and compressing the resultant admixture into tablets. Advantages of wet granulation include improvement of the cohesiveness and compressibility of powders, a good particle size distribution suitable for compression, reduction of dust and airborne contamination, and prevention of the segregation of components.

In dry granulation, the ingredients are weighed, mixed and compacted, such as by roller compaction, and subsequently broken up or screened. The screened granules are lubricated and compressed into tablets. Since no liquid binder solution is used for agglomeration, the powder mixture which is granulated in dry form must comprise at least one dry binding agent such as microcrystalline cellulose, polyvinyl pyrrolidone, or a co-processed mixture of lactose and microcrystalline cellulose. One of the advantages of dry granulation methods is that they may be suitable for the processing of sensitive materials, such as moisture- or heat-sensitive ingredients, as no water is added during the process and no heating is required for drying the granules.

Direct compression involves the compaction of powder mixtures into tablets without prior granulation. This method is potentially cost-effective as it avoids the sequence of process steps involved in the preparation of granules, and it is also suitable for the processing of sensitive active compounds. The presence of a dry binding agent or compression aid in the formulation is usually required or desirable for achieving useful tablet strengths. However, direct compression may not always be possible. For example, certain powder mixtures do not exhibit a sufficient product flow on tablet presses or do not yield suitable tablet physical characteristics, so that in these cases the use of granulation is preferred.

It has been found that powder mixtures of a water-swellable polymer, a dry binding agent or compression aid, and a water-soluble salt of neramexane and, optionally, further excipients, are suitable for direct compression. Typically, the three components (i.e. water-sellable polymer, dry binding agent, and neramexane salt) represent at least about 75 wt.-% of the powder mixture, and the optional further excipients represent no more than about 25 wt.-%. In another embodiment, the water-swellable polymer (or mixture of water-swellable polymers), the dry binding agent or compression aid (or mixture of more than one member of this class), and the active compound together represent at least about 85 wt.-% of the matrix formulation, such as from about 85 wt.-% to abut 99.9 wt.-%, or from about 90 wt.-% to about 99.5 wt.-%. In a further embodiment, they constitute from about 95 wt.-% to about 99 wt.-% of the matrix.

According to another embodiment, the invention involves the direct compression of a powder mixture comprising hydroxypropyl methylcellulose, such as Methocel K 100M CR, microcrystalline cellulose, such as Avicel PH 102, and neramexane mesylate. Typically, each of these three components represents from about 10 wt.-% to about 50 wt.-% of the powder that is compacted to form the matrix. In another embodiment, the ratio of neramexane mesylate to the other two components together is from about 1:1 to about 1: 5, and more preferably from about 1:1 to about 1:3, such as about 1:2.

The powder mixture may comprise one or more further excipients. Among the further excipients are members of the classes of lubricants, such as magnesium stearate, stearic acid, calcium stearate, sodium stearyl fumarate, mineral oil, hydrogenated vegetable oil, and polyethylene glycol; and glidants, such as colloidal silicon dioxide, starch, calcium or magnesium stearate, and talc.

The lubricant is typically used at a level from about 0.1 wt. % to about 2 wt.-%, relative to the weight of the matrix. A representative lubricant is magnesium stearate, which also has some glidant properties. If magnesium stearate is selected, a useful content range is from about 0.2 wt.-% to about 1.5 wt.-%, in particular from about 0.25 wt.-% to about 1 wt.-%.

Similarly, the amount of glidant should be selected at a relatively low level, such as below about 5 wt.-%. Among the representative glidants are colloidal silicon dioxide and talc. If one or both of these glidants are incorporated, the glidant content in the matrix is typically in the region from about 0.25 wt.-% to about 2.5 wt.-%, or from about 0.5 wt.-% to about 1.5 wt.-%.

Such matrix dosage forms have been surprisingly found to exhibit extremely favorable tablet properties. For example, it was found that the release profile of the active compound was relatively independent of the compression force, at least over a broad range of practically useful compression forces. Mixtures of Methocel K 100M CR, Avicel PH 102, neramexane mesylate, magnesium stearate and colloidal silicon could be tabletted on a standard rotary tablet press using main compression forces ranging from about 5 kN to about 21 kN. The resulting tablets varied substantially in their tensile strength, from about 30 N to about 100 N, with higher compression forces leading to harder tablets. However, the dissolution profiles of these tablets were substantially similar, even when the hardest tablets were compared to the softest tablets with identical composition, indicating a remarkably robust formulation. In particular, it was found that within the hardness range from about 40 N to about 80 N, the dissolution profiles are substantially independent of the hardness or tensile strength of the tablets. The tensile strength may vary from about 30 N to about 500 N, such as from about 40 N to about 300 N or from about 50 N to about 200 N. Moreover, the tensile strength of the film-coated tablets may be above 120 N.

Another highly favorable feature of the tablets of the invention is that, while neramexane mesylate is an acid-addition salt of neramexane of which a pH-dependent dissolution behavior may be expected, the above-identified matrices release the active compound relatively independently of the pH of the dissolution medium.

As used herein, the terms “relatively independent” and “substantially independent” mean that the in-vitro release profiles of two tablets or matrices do not differ by more than about 10% of the incorporated dose at any point of time after the initial phase of drug release (0 to 1 hour).

According to a further embodiment, the dosage form of the present invention is designed as a compressed matrix which is coated with a coating, such as a sugar or polymeric coating, to provide taste masking of an active compound which typically has a poor taste.

As used herein, a taste masking coating is a coating which does not substantially influence the drug release profile of the modified release matrix. In other words, except perhaps in the initial phase of drug release, there will be no substantial difference at any time between the dose fraction released from an uncoated matrix and an identically formulated and processed matrix which has a taste masking coating. Again, a substantial difference is understood as a difference of 10% or more of the dose of active compound incorporated in the matrix. It is believed that the greatest impact of a taste masking coating on the shape of the drug release profile is in the initial phase of drug release, such as during the first 15 or 30 minutes, which is not relevant to the overall release characteristics of a modified release dosage form.

Typically, the coating of the matrix is a polymeric film coating. Film compositions suitable for taste masking are widely known in the field of pharmaceuticals, and they may be based on various types of polymers. Typically, a taste masking coating prevents the direct contact of the active compound with saliva during administration, and rapidly dissolves or disintegrates after the dosage form has been swallowed.

Suitable polymers for such coatings include e.g. cationic methacrylate copolymers, such as dimethylaminoethyl methacrylate methylmethacrylate copolymer (DMA-MMA), which is insoluble in aqueous media above pH 5 (such as saliva), but dissolves in acidic media (such as gastric fluid). Other potentially suitable polymers include hydroxypropyl cellulose, hydroxypropyl methylcellulose, hydroxyethyl methylcellulose, methacrylic acid copolymers other than DMA-MMA, polyvinyl alcohol-polyethylene glycol copolymer, ethyl acrylate-methyl methacrylate copolymer, polyvinyl alcohol, carrageenan, and mixtures thereof.

The coating composition may comprise further excipients to improve the properties of the coating or its processability, such as one or more excipient selected from the classes of plasticizers, stabilizers, pigments, coloring agents, dispersing agents, surfactants, sugars, fillers, antiadhesives, water vapor permeability-modifying agents etc. Commercially available coating compositions often represent premixes of one or more film-forming polymers and at least one further excipient. Useful commercial coating compositions include the water-soluble grades of Sepifilm, such as Sepifilm 002, Sepifilm 003, Sepifilm 752, Sepifilm LP grades including Sepifilm LP 770; water-soluble grades of Kollicoat, such as Kollicoat IR and Kollicoat Protect; furthermore Opadry, virtually all grades of Instacoat, LustreClear, and similar products.

The taste masking coating may have other functions. For example, the coating potentially improves the mechanical and even the chemical stability of the matrix tablet, and it may also improve the appearance of the tablet, its appeal to the patient, swallowability and other features.

The coatings may be applied to the matrices by any conventional technique and equipment, such as by pan coating or fluid-bed coating. Typically, an aqueous, hydroalcoholic, or organic liquid comprising the dispersed or dissolved film-forming polymer(s) and any optional further excipients is atomized and deposited on pre-formed tablet cores which have optionally been de-dusted, under a continuous flow of warm air to dry the coating composition on the tablet cores.

In accordance with the present invention, a modified release dosage form is provided for the once daily administration of neramexane or a pharmaceutically acceptable salt thereof, to a human or animal subject. The neramexane formulations of the invention are suitable for the treatment of CNS diseases, including but not limited to the treatment of Alzheimer's disease, Parkinson's disease, AIDS dementia, neuropathic pain, diabetic neuropathic pain, cerebral ischemia, epilepsy, glaucoma, hepatic encephalopathy, multiple sclerosis, stroke, depression, tardive dyskinesia, amyotrophic lateral sclerosis, irritable bowel syndrome, appetite disorders, binge eating disorders, autism, attention deficit syndrome, attention deficit hyperactivity disorder, bipolar disorder, tinnitus, mycosis, psoriasis, malaria, Borna virus, and Hepatitis C. Additional pathologies for treatment of which neramexane is suitable are disclosed in the art. Of particular interest is the ability to provide uninterrupted pain relief. Accordingly, the present invention further provides a method for the therapeutic or prophylactic treatment of CNS disorders in a human or animal subject, the method including administering to the subject, a dosage form in accordance with the present invention.

A “therapeutically effective amount” means the amount of a compound that, when administered to a mammal for treating a state, disorder or condition is sufficient to effect such treatment. The “therapeutically effective amount” will vary depending on the compound, the disease and its severity and the age, weight, physical condition and responsiveness of the mammal to be treated. According to the instant invention, in one embodiment, a therapeutically effective amount of neramexane is an amount effective to treat CNS disorders, including Alzheimer's disease or Parkinson's disease. In another embodiment, a therapeutically effective amount is an amount effective to treat neuropathic pain, or other painful conditions such as visceral hypersensitivity. Other uses include, but are not limited to, the treatment of dementia and depression. The effective amount of the drug for pharmacological action, and therefore the tablet strength, depends on the disease itself.

As used herein, the term “treat” is used herein to mean to relieve or alleviate at least one symptom of a disease in a subject, including for example, pain, Alzheimer's disease, vascular dementia, or Parkinson's disease. The term “treat” may mean to relieve or alleviate the intensity and/or duration of a manifestation of disease experienced by a subject in response to a given stimulus (e.g., pressure, tissue injury, cold temperature, etc.). For example, in relation to dementia, the term “treat” may mean to relieve or alleviate cognitive impairment (such as impairment of memory and/or orientation) or impairment of global functioning (activities of daily living, ADL) and/or slow down or reverse the progressive deterioration in ADL or cognitive impairment. Within the meaning of the present invention, the term “treat” also denotes to arrest, delay the onset (i.e., the period prior to clinical manifestation of a disease) and/or reduce the risk of developing or worsening a disease. The term “protect” is used herein to mean prevent delay or treat, or all, as appropriate, development or continuance or aggravation of a disease in a subject. Within the meaning of the present invention, the dementia is associated with a CNS disorder, including without limitation neurodegenerative diseases such as Alzheimer's disease (AD), Down's Syndrome and cerebrovascular dementia (VaD).

The invention is further illustrated by the following examples, which should however not be interpreted as to limit the scope of the invention.

### EXAMPLES

### Example 1

### Preparation of Neramexane Modified Release Matrix Tablets

Matrix tablets comprising approx. 25 mg or 50 mg or 75 mg or 100 mg neramexane mesylate are prepared as follows. The appropriate amounts of neramexane mesylate, hydroxypropyl methyl cellulose (HPMC, here: Methocel K 100M CR), microcrystalline cellulose (MCC, here: Avicel PH 102), magnesium stearate and colloidal silicon dioxide (SiO₂, here: Aerosil 200) are weighed, and blended using a free fall blender (Bohle PTM 200). Alternatively the appropriate amounts of neramexane mesylate, hydroxypropyl methyl cellulose, microcrystalline cellulose, magnesium stearate and colloidal silicon dioxide are sieved before being blended using the free fall blender. The appropriate amounts for each batch are calculated according to the target contents per dosage unit as given in Table 1. The optical characterization of the powder blends show no lack of homogeneity like flakes, lumps or segregation tendencies. All blends show good powder flow properties and are freely flowing. The bulk and tapped densities of all blends are not significantly different.

TABLE 1

| Ingredi- | Formulation | Formulation | Formulation | Formulation |
|---|---|---|---|---|
| ent | A [mg/tablet] | B [mg/tablet] | C [mg/tablet] | D [mg/tablet] |
| Nera- | 50.0 | 50.0 | 50.0 | 50.0 |
| mexane | | | | |
| mesylate | | | | |
| HPMC | 25.0 | 50.0 | 75.0 | 200 |
| MCC | 72.75 | 47.75 | 22.75 | 45.50 |
| SiO₂ | 1.5 | 1.5 | 1.5 | 3.0 |
| Magne- | 0.75 | 0.75 | 0.75 | 1.50 |
| sium | | | | |
| stearate | | | | |
| Total | 150.0 | 150.0 | 150.0 | 300.0 |

| Ingredi- | Formulation | Formulation | Formulation | Formulation |
|---|---|---|---|---|
| ent | E [mg/tablet] | F [mg/tablet] | G [mg/tablet] | H [mg/tablet] |
| Nera- | 25.0 | 25.0 | 75.0 | 100.0 |
| mexane | | | | |
| mesylate | | | | |
| HPMC | 50.0 | 125.0 | 300.0 | 400.0 |
| MCC | 48.125 | 47.0 | 68.25 | 91.0 |
| SiO₂ | 1.25 | 2.0 | 4.5 | 6.0 |
| Magne- | 0.625 | 1.0 | 2.25 | 3.0 |
| sium | | | | |
| stearate | | | | |
| Total | 125.0 mg | 200 mg | 450.0 mg | 600.0 mg |

The powders are separately compressed into biconvex matrix tablets using a conventional rotary tabletting press applying main compression forces of approx. 10 to 20 kN. For example, after production, the mean content of neramexane mesylate for the 50 mg formulations is found to be between 47.5 and 52.5 mg/tablet, such as between 50 and 52 mg/tablet, for all batches, with a content uniformity meeting the requirements of the European Pharmacopeia and the US Pharmacopeia.

### Example 2

### Coating of Neramexane Modified Release Matrix Tablets

Matrix tablets prepared according to Example 1 are coated with a white, water-soluble coating composition of Sepifilm LP 770 white, using a perforated or non perforated standard pan coater with air control. Prior to coating, the tablets are weighed and de-dusted. Subsequently, the coating dispersion is sprayed onto the tablets using a 1.0 mm nozzle. The temperature of the tablet cores at the time of coating is between 34 and 39° C. The inlet temperature is between 59 and 64° C., and the spray rate is approximately 40-53 g/min. Spraying is continued until the weight gain of the tablets is about 4%. The optical appearance of the coated tablets is very good. There appears to be no sticking, the surface is smooth, brilliant and very homogeneous without any cracks or damages.

### Example 3

### Drug Release from Modified Release Matrix Tablets

Tablets are prepared according to Example 1 their drug release profiles are determined using a basket-type dissolution apparatus according to USP XXVII, an agitation rate of 100 rpm, and phosphate buffer of pH 6.8 as dissolution medium. At certain time intervals, samples of the dissolution medium are withdrawn and analyzed for their content of neramexane. For the Formulations A-D the results are summarized in Table 2.

TABLE 2

| Time | Formulation A | Formulation B | Formulation C | Formulation D |
|---|---|---|---|---|
| [min] | [% released] | [% released] | [% released] | [% released] |
| 60 | 42 | 34 | 33 | 25 |
| 120 | 61 | 50 | 48 | 36 |
| 180 | 75 | 64 | 61 | 45 |
| 240 | 85 | 74 | 73 | 52 |
| 300 | 91 | 82 | 79 | 59 |
| 360 | 95 | 88 | 85 | 65 |
| 480 | 99 | 95 | 94 | 75 |

The results demonstrate how the drug release profile of the dosage form of the invention may be fine-tuned simply by varying the relative content of the water-swellable polymer in the matrix tablets.

### Example 4

### Preparation of Neramexane Matrix Tablets with Different Tensile Strengths

Neramexane modified release matrix tablets are prepared according to Example 1, having the same qualitative and quantitative composition as formulation B (Table 1), except that the main compression force is varied: One batch of tablets (B-soft) is compressed with a low force of approx. 5 kN, and a second batch (B-hard) with a high force of approx. 21 kN. The hardness or tensile strength of the resulting tablets varies significantly: Tablets of batch B-soft soft have a hardness ranging from 33 to 38 N, whereas batch B-hard have a hardness ranging from 85 to 96 N.

### Example 5

### Drug Release from Neramexane Tablets with Different Tensile Strengths

Tables prepared according to Example 4 are tested for their drug release properties as described in Example 3. The results, which demonstrate a remarkably robust formulation, are summarized in Table 3.

TABLE 3

| Time [min] | Batch B-soft [% released] | Batch B-hard [% released] |
|---|---|---|
| 60 | 39 | 37 |
| 120 | 57 | 55 |
| 180 | 69 | 67 |
| 240 | 78 | 76 |
| 300 | 85 | 84 |
| 360 | 90 | 90 |
| 480 | 96 | 97 |

### Example 6

### pH-Independent Drug Release from Neramexane Matrix Tablets

Neramexane modified release matrix tablets according to formulation B are prepared as described in Example 1. Subsequently, the tablets are film-coated as described in Example 2. The drug release profiles of the film-coated tablets are investigated with a basket apparatus and an agitation rate of 100 rpm at pH 4.5 and pH 7.4.

The shapes of the release profiles are very similar, and only small differences between the profiles are observed, indicating the remarkably robust formulation and its very low pH dependence over a broad range of pH values. In fact, the largest observed difference in a released dose fraction at any point of time between two pH conditions is only 6%. The results are summarized in Table 4.

TABLE 4

| | pH 1.2 | pH 4.5 | pH 7.4 |
|---|---|---|---|
| Time [min] | [% released] | [% released] | [% released] |
| 60 | 35 | 36 | 33 |
| 120 | 53 | 54 | 50 |
| 180 | 67 | 68 | 63 |
| 240 | 76 | 77 | 72 |
| 300 | 84 | 85 | 79 |
| 360 | 88 | 91 | 85 |
| 480 | 94 | 98 | 92 |

### Example 7

### Calculation of Plasma Profiles Achievable with Neramexane Matrix Tablets

Neramexane plasma concentrations over time are determined after the administration of a single dose of 25 mg neramexane mesylate in an immediate release formulation to a number of volunteers. From the plasma concentration profiles, the mean absorption and elimination rate constants are calculated. Using these data, the expected neramexane plasma concentration profiles at steady state are calculated for the following therapeutic regimen: (a) twice-daily administration of 25 mg neramexane mesylate as immediate release formulation, (b) once-daily administration of 50 mg neramexane mesylate as immediate release formulation, and (c) once-daily administration of 50 mg neramexane mesylate in the form of formulation B of Example 1. From the simulated plasma profiles, the respective fluctuation indices are calculated.

It is found that therapeutic regimen (a) is associated with a fluctuation index of approx. 0.22, regimen (b) with a fluctuation index of approx. 0.47, and regimen (c) with a fluctuation index of approx. 0.33. These results reflect the fact that regimen (a) and (c), but not regimen (b) is considered acceptable in terms of risk of side effects and risk of sub-therapeutic trough concentrations. While regimen (a) involves only small fluctuations of plasma concentrations, it requires twice-daily dosing, which is considered less convenient than the once-daily regimen (c), at least for a continuous therapy.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

It is further to be understood that all values are approximate, and are provided for description.

Patents, patent applications, publications, product descriptions, and protocols are cited throughout this application, the disclosures of which are incorporated herein by reference in their entireties for all purposes.

1. An oral modified release dosage form comprising: a therapeutically effective amount of an active compound selected from neramexane, isomers thereof and water soluble and pharmaceutically acceptable salts, solvates, conjugates, prodrugs and derivatives thereof, and at least one release-controlling excipient, wherein the content of the excipient is selected to achieve an in vitro active compound release profile characterized by a dissolution time of at least about 1 hour for a fraction of about 10 to about 70 wt.-% of the amount of the active compound.

2. The dosage form of claim 1, wherein the in vitro active compound release profile is characterized by a dissolution time of at least about 1 hour for a fraction of 40 wt.-% of the amount of the active compound.

3. The dosage form of claim 1, wherein the in vitro active compound release profile is characterized by a dissolution time of at least about 1 hour for a fraction of 50 wt.-% of the amount of the active compound.

4. The dosage form of claim 1, wherein the in vitro active compound release profile is characterized by a dissolution time of at least about 1 hour for a fraction of 60 wt.-% of the amount of the active compound.

5. The dosage form of claim 1, wherein the in vitro active compound release profile is characterized by a dissolution time ranging from about 1 to about 8 hours for a fraction of about 10 to about 70 wt.-% of the amount of the active compound.

6. The dosage form of claim 1, wherein the in vitro active compound release profile is characterized by a dissolution time ranging from about 1 hour to about 3 hours for a fraction of 50 wt.-% of the amount of the active compound.

7. The dosage form of claim 1, wherein the in vitro active compound release profile is characterized by a dissolution time of 4 hours for a fraction ranging from about 50 wt.-% to about 95 wt.-% of the amount of the active compound.

8. The dosage form of claim 7, wherein the in vitro active compound release profile is characterized by a dissolution time of 4 hours for a fraction ranging from about 65 wt.-% to about 95 wt.-% of the amount of the active compound.

9. The dosage form of claim 7, wherein the in vitro active compound release profile is characterized by a dissolution time of 4 hours for a fraction ranging from about 55 wt.-% to about 85 wt.-% of the amount of the active compound.

10. The dosage form of claim 9, wherein the in vitro active compound release profile is characterized by a dissolution time of 4 hours for a fraction ranging from about 70 wt.-% to about 85 wt.-% of the amount of the active compound.

11. The dosage form of claim 1, wherein the active compound is neramexane mesylate.

12. The dosage form of claim 1, wherein the therapeutically effective amount of the active compound is in the range of about 5 mg to about 150 mg for neramexane mesylate or an equimolar amount for neramexane, another pharmaceutically acceptable salt, a solvate, an isomer, a conjugate, a prodrug or a derivative thereof.

13. The dosage form of claim 12, wherein the therapeutically effective amount of the active compound is in the range of about 5 mg to about 100 mg for neramexane mesylate or an equimolar amount for neramexane, another pharmaceutically acceptable salt, a solvate, an isomer, a conjugate, a prodrug or a derivative thereof.

14. The dosage form of claim 13, wherein the therapeutically effective amount of the active compound is in the range of about 5 mg to about 50 mg for neramexane mesylate or an equimolar amount for neramexane, another pharmaceutically acceptable salt, a solvate, an isomer, a conjugate, a prodrug or a derivative thereof.

15. The dosage form of claim 14, wherein the therapeutically effective amount of the active compound is in the range of about 5 mg to about 40 mg for neramexane mesylate or an equimolar amount for neramexane, another pharmaceutically acceptable salt, a solvate, an isomer, a conjugate, a prodrug or a derivative thereof.

16. The dosage form of claim 15, wherein the therapeutically effective amount of the active compound is in the range of about 10 mg to about 30 mg for neramexane mesylate or an equimolar amount for neramexane, another pharmaceutically acceptable salt, a solvate, an isomer, a conjugate, a prodrug or a derivative thereof.

17. The dosage form of claim 14, wherein the therapeutically effective amount of the active compound is selected from 5 mg, 6.25 mg, 7.5 mg, 10 mg, 12.5 mg, 15 mg, 17.5 mg, 20 mg, 22.5 mg, 25 mg, 27.5 mg, 30 mg, 32.5 mg, 35 mg, 37.5 mg, 40 mg, 42.5 mg, 45 mg, 47.5 mg and 50 mg for neramexane mesylate or an equimolar amount for neramexane, another pharmaceutically acceptable salt, a solvate, an isomer, a conjugate, a prodrug or a derivative thereof.

18. The dosage form of claim 1, wherein the active compound is dispersed within a solid matrix formed by the at least one release-controlling excipient and, optionally, one or more further excipients.

19. The dosage form of claim 18, wherein the content of the release-controlling excipient in the solid matrix ranges from about 10 wt.-% to about 80 wt.-%.

20. The dosage form of claim 18, further comprising one or more excipients selected from dry binding agents, lubricants, and glidants.

21. The dosage form of claim 20, wherein the dry binding agent is selected from lactose, lactose monohydrate, calcium phosphate, calcium hydrogen phosphate, calcium sulfate, sucrose, dextrose, mannitol, sorbitol, cellulose, and microcrystalline cellulose.

22. The dosage form of claim 20, wherein the lubricant is selected from magnesium stearate, stearic acid, calcium stearate, sodium stearyl fumarate, mineral oil, hydrogenated vegetable oil, and polyethylene glycol.

23. The dosage form of claim 20, wherein the glidant is selected from colloidal silicon dioxide, starch, calcium or magnesium stearate, and talc.

24. The dosage form of claim 18, wherein the solid matrix is in the form of a compressed tablet.

25. The dosage form of claim 24, wherein the compressed tablet is a directly compressed tablet.

26. The dosage form of claim 24, wherein the compressed tablet is coated with a taste-masking coating.

27. The dosage form of claim 26, wherein the coating is polymeric.

28. The dosage form of claim 1, wherein the release-controlling excipient is selected to achieve an in vitro active compound dissolution profile which is substantially independent of the pH of the dissolution medium.

29. The dosage form of claim 1, wherein the release-controlling excipient is a water-swellable polymer.

30. The dosage form of claim 29, wherein the water-swellable polymer is selected from methylcellulose, hydroxymethylcellulose, hydroxyethylcellulose, hydroxypropylcellulose, hydroxybutylcellulose, hydroxyethyl methylcellulose, hydroxypropyl methylcellulose, carboxymethylcellulose, carboxymethyl ethylcellulose, alginate, carrageen, galactomannan, tragacanth, agar, acacia, guar gum, xanthan gum, pectin, carboxymethyl amylopectin, chitosan, polyacrylate, polymethacrylate, methacrylate copolymer, polyvinyl alcohol, polyvinylpyrrolidone, polyvinylpyrrolidone vinyl acetate copolymer, and mixtures thereof.

31. The dosage form of claim 29, wherein the water-swellable polymer is a non-ionic cellulose ether.

32. The dosage form of claim 31, wherein the water-swellable polymer is hydroxypropyl methylcellulose.

33. The dosage form of claim 29, wherein the weight ratio of the active compound to the water-swellable polymer is in the range from about 10:1 to about 1:10.

34. The dosage form of claim 33, wherein the weight ratio of the active compound to the water-swellable polymer is in the range from about 4:1 to about 1:4.

35. The dosage form of claim 34, wherein the weight ratio of the active compound to the water-swellable polymer is in the range from about 2:1 to about 1:2.

36. The dosage form of claim 1, wherein the active compound is dispersed in a matrix in the form of a compressed tablet, and wherein the release-controlling excipient is selected to achieve an in vitro active compound dissolution profile which is substantially independent of the hardness of the compressed tablet, wherein the hardness is within the range from about 40 N to about 80 N.

37. An oral modified release dosage form comprising: a therapeutically effective amount of an active compound selected from neramexane and pharmaceutically acceptable salts, solvates, isomers, conjugates, prodrugs and derivatives thereof; and at least one release-controlling excipient, wherein the release-controlling excipient is selected to achieve a fluctuation index of neramexane plasma concentration of about 0.4 or less upon once-daily administration in steady state.

38. A method of treating a living animal body, including a human, afflicted with a condition selected from mild, moderate, or severe Alzheimer's dementia, and neuropathic pain, diabetic neuropathic pain, amyotrophic lateral sclerosis, multiple sclerosis, irritable bowel syndrome, appetite disorders, obesity, binge eating disorders, autism, attention deficit syndrome, attention deficit hyperactivity disorder, bipolar disorder, tinnitus, mycosis, or psoriasis, comprising the step of administering to the living animal body, including a human, the dosage form of claim 1.

39. The method of claim 38, wherein the condition is selected from mild, moderate, or severe Alzheimer's dementia, or neuropathic pain.

40. A method of treating a living animal body, including a human, afflicted with a condition selected from mild, moderate, or severe Alzheimer's dementia, and neuropathic pain, diabetic neuropathic pain, amyotrophic lateral sclerosis, multiple sclerosis, irritable bowel syndrome, appetite disorders, obesity, binge eating disorders, autism, attention deficit syndrome, attention deficit hyperactivity disorder, bipolar disorder, tinnitus, mycosis, or psoriasis, comprising the step of administering to the living animal body, including a human, the dosage form of claim 37.

41. The method of claim 40, wherein the condition is selected from mild, moderate, or severe Alzheimer's dementia, or neuropathic pain.

42. A method of treating a living animal body, including a human, afflicted with a condition associated with cognitive impairment comprising the step of administering to the living animal body, including a human, the dosage form of claim 14.

43. The method of claim 42, wherein the condition associated with cognitive impairment is selected from dementia; neurodegenerative dementia; mild, moderate and severe Alzheimer's dementia; Parkinson's dementia; AIDS dementia; schizophrenia; attention deficit syndrome; attention deficit hyperactivity disorder; Korsakoff syndrome; cerebrovascular dementia; frontotemporal dementia; autism; corticobasal degeneration; Lewis body disease; mild cognitive impairment; dementia due to inflammation or infection; multiple sclerosis; or amyotrophic lateral sclerosis.

44. The method of claim 42, wherein the dosage form is administered once daily.
